(19) United States
(12) Patent Application Publication
(10) Pub. No.: US 2015/0272105 A1
(43) Pub. Date: Oct. 1, 2015

(54) Real Time Sensing of Pests

(71) Applicants: John PETERSON, Jackson, MN (US); AGCO CORPORATION, Duluth, GA (US)

(72) Inventor: John Peterson, Jackson, MN (US)

(73) Assignee: AGCO Corporation, Duluth, GA (US)

(21) Appl. No.: 14/432,286

(22) PCT Filed: Sep. 27, 2013
(86) PCT No.: PCT/US13/62082

§ 371 Date: Mar. 30, 2015

Related U.S. Application Data
(60) Provisional application No. 61/707,226, filed on Sep. 28, 2012.

(51) Int. Cl. A01M 1/02; G01N 33/00; G01N 33/02; A01M 7/00

(52) U.S. Cl. CPC A01M 1/026; A01M 7/0089; G01N 33/0009; G01N 33/025

(57) ABSTRACT

A real time sensing method, comprising transporting a portable sensing system on a field comprising crops; receiving at an air intake system of the portable sensing system one or more organic chemical compounds emitted from the crops in the field; detecting by the portable sensing system in real time a chemical of interest from the received one or more organic compounds; and providing by the sensing system feedback of the detection.

### CROSS REFERENCE TO RELATED APPLICATION

This application claims the benefit of U.S. Provisional Application No. 61/707,226, filed Sep. 28, 2012, which is hereby incorporated by reference in its entirety.

### TECHNICAL FIELD

The present disclosure is generally related to crop protection.

### BACKGROUND

Currently, field scouting for pests is a time consuming and labor intensive activity. In general, field scouting may involve walking through a field and stopping at one or more locations to make and record observations. Proper examination of the field may help to accurately identify yield-limiting problems during the growing season when they can often be corrected so that full yield potential can be preserved. Further, the recorded scouting information may be useful for future reference to avoid problems in subsequent years. For example, a pest such as soybean cyst nematode impacts both crop rotation and variety selection when soybeans are grown again in the same field. Accurate records may aid in the decisions required to help manage this pest.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1A is a schematic diagram that illustrates a person performing field scouting using an embodiment of a real time sensing system.

FIG. 1B is a schematic diagram that illustrates an embodiment of a real time sensing system coupled to an agricultural machine.

FIG. 2 is a block diagram of an embodiment of an example real time sensing system.

FIG. 3 is a schematic diagram that illustrates sensor outputs before and after pest treatment.

FIG. 4 is a flow diagram of an embodiment of an example real time sensing method.

FIG. 5 is a flow diagram of another embodiment of an example real time sensing method.

### DESCRIPTION OF EXAMPLE EMBODIMENTS

### Overview

In one embodiment, a real time sensing method, comprising transporting a portable sensing system on a field comprising crops; receiving at an air intake system of the portable sensing system one or more organic chemical compounds emitted from the crops in the field; detecting by the portable sensing system in real time a chemical of interest from the received one or more organic compounds; and providing by the sensing system feedback of the detection.

### Detailed Description

Certain embodiments of real time sensing systems and methods are disclosed that include sensors that detect chemical compounds given off by crops and/or other vegetation in a field in response to pest infestation. For instance, when pests such as soybean aphids attack a field, it is known that plants emit certain organic compounds as a defense mechanism. In one embodiment, a real time sensing system is transported on a field (e.g., secured on an agricultural machine, or secured on a person and/or article of clothing of that person, such as a farmer or farm employee). Using the agricultural machine as an illustrative, non-limiting example, when the real time sensing system enters the field, one or more chemical sensors (e.g., a sensor array) is initiated and begins to collect data for processing in a processor. If the data received is “fingerprinted” as a chemical of interest, a feedback mechanism is established (e.g., alerting personnel, prompting a spray operation to disperse insecticides, etc.). Based on the feedback mechanism, the field can be treated and the chemical sensor may be used to evaluate the effectiveness of the treatment.

Having summarized certain features of real time sensing systems of the present disclosure, reference will now be made in detail to the description of the disclosure as illustrated in the drawings. While the disclosure will be described in connection with these drawings, there is no intent to limit it to the embodiment or embodiments disclosed herein. Further, although the description identifies or describes specifics of one or more embodiments, such specifics are not necessarily part of every embodiment, nor are all various stated advantages necessarily associated with a single embodiment or all embodiments. On the contrary, the intent is to cover all alternatives, modifications and equivalents included within the spirit and scope of the disclosure as defined by the appended claims. Further, it should be appreciated in the context of the present disclosure that the claims are not necessarily limited to the particular embodiments set out in the description.

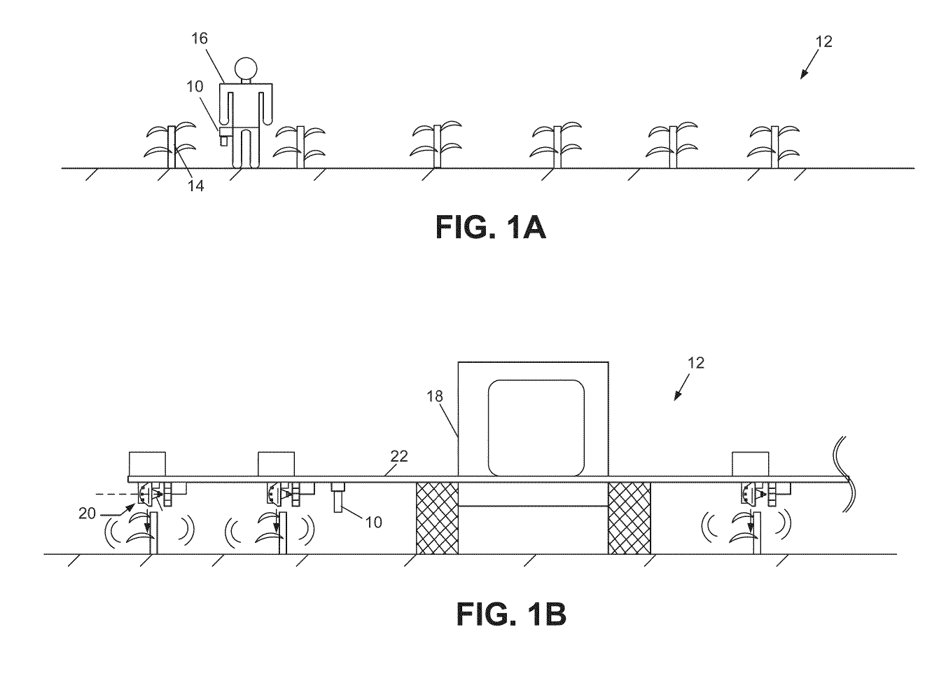

Referring now to FIG. 1A, shown is a schematic diagram of one example field scouting implementation where one or more real time sensing systems, such as real time sensing system 10, is transported in a field 12 comprising a plurality of crops 14 and/or other vegetation. The real time sensing system 10 may be equipped with one or more chemical sensors, including chemicapacitor-based and/or chemiresistor-based sensors, a processor, an air induction system, and data transfer functionality. The real time sensing system 10 may be carried by a person 16, such as in his or her hand, or secured to and/or otherwise carried in an article of clothing, such as a belt or pocket. The person 16 is performing field scouting to determine whether there are pests that have infiltrated the field 12. Upon detection of a chemical of interest (e.g., an organic chemical compound emitted by the crops 14 in response to pest infestation), the real time sensing system 10 may provide feedback of the detection/identification in the form of a visual (e.g., blinking or otherwise activated light, such as a light-emitting diode (LED) disposed on the field sensing device), audio (e.g., alarm), and/or tactile (e.g., vibrate, buzz, etc.) signal (directly via the real time sensing system 10 or cause to occur in another device) to the person 16. In some embodiments, the real time sensing system 10 may communicate the detection over a wireless network (e.g., via an RF channel or carrier network) to, for instance, a device (e.g., computer and/or communications device) in farm management office. In some embodiments, plural personnel may be in the field 12 performing field scouting, each equipped with a real time sensing system 10. The plural real time sensing systems 10 may communicate with each other in peer-to-peer fashion to enable sharing of data, and hence a determination of the extent or scope of pest infestation. In some embodiments, the data from each of the real time sensing systems 10 in a given field may be communicated to a central office computer (e.g., over a network, or in some embodiments, later downloaded, such as via a USB connector on the real time sensing system) for determination of the location and/or extent of pest infestation.

FIG. 1B provides a schematic diagram of another example field scouting implementation, where the real time sensing system 10 (or a plurality of real time sensing systems) may be coupled to an agricultural machine, such as a sprayer machine 18 (shown in rear end, elevation view and moving into the page) that comprises plural sprayers 20 (e.g., controlled droplet applicators, hydraulic sprayers, etc. with or without air flow assist devices, such as fans) along a boom 22 (partially shown in FIG. 1B) that may be activated in response to feedback from the real time sensing system 10. For instance, the real time sensing system 10 may cause a control signal to be transmitted to an actuator of one of the sprayers 20 (e.g., a sprayer that is local to the pest infestation for selective treatment) or plural sprayers (e.g., to cover an area local to the detected area(s), with the programmed assumption, or based on peer-feedback, that infestation is more widespread than a single, localized location). In some embodiments, an indication of the extent or scope of infestation may be determined based on the strength (e.g., surpassing a given threshold sensor output) or quantity of the matching fingerprints (e.g., with a reference fingerprint for a given chemical of interest), such as where there are plural real time sensing systems 10 located along the boom 22. In one embodiment, the real time sensing systems 10 may communicate the data to a central computer (e.g., located in the cab of the sprayer machine 18 or at a central office), and a comparison of the data may be made to determine relative strengths (e.g., concentrations) of the emitted chemical compounds among the locations corresponding to the plural real time sensing systems 10. In some embodiments, such comparisons may be made at each real time sensing system 10 located along the boom 22 based on peer-to-peer processing and communication of data.

Note that although a sprayer machine 18 is provided as one example machine to be used in transporting the real time sensing system 10, other machines (e.g., tractors, ATVs, etc.) may be used in some embodiments. Further, although an agricultural environment is depicted in FIGS. 1A-1B, it should be appreciated that other environments are contemplated to be within the scope of application, including residential, recreational, and/or commercial property.

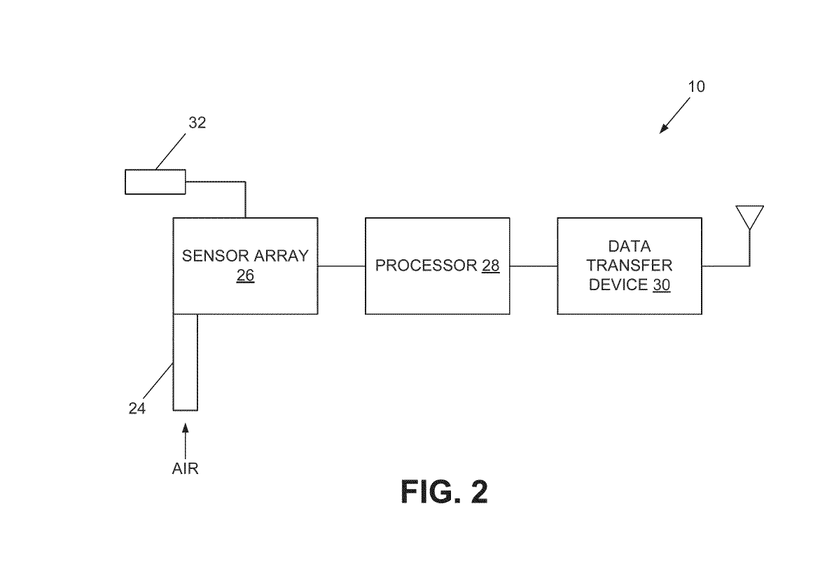

Having described some example field scouting implementations using the real time sensing systems 10, attention is directed to FIG. 2, which provides a block diagram illustration of an embodiment of the real time sensing system 10. It should be appreciated that the example real time sensing system 10 depicted in FIG. 2 is one example among other possible examples, where fewer or a greater number of components may be used. In one embodiment, the real time sensing system 10 may be integrated in a single package, such as a handheld unit. In some embodiments, the real time sensing system 10 may include nontechnology, MEMs (micro-electro-mechanical) technology, or the like. In some embodiments, functionality of the real time sensing system 10 may be distributed over plural separate units. Referring to FIG. 2, the real time sensing system 10 comprises an air induction system 24, a sensor array 26, a processor 28, and a data transfer device 30. Note that some embodiments of a real time sensing system 10 may include additional or fewer components than those shown in FIG. 2. The air induction system 24 may operate according to a passive mechanism for air ingress or an active, air-inducing mechanism (e.g., such as through the use of a micropump, vacuum pump, etc.). The air induction system 24 receives, or draws in, air comprising one or more organic compounds (e.g., volatile compounds), such as organic compounds emitted from pest-infested crops and/or vegetation.

The sensor array 26 may comprise one or more chemical sensors, such as chemicapacitor and/or chemiresistor sensors. Chemical sensor technology is known in the art, such as those manufactured by Seacoast Science, Inc. (e.g., the SC-210 series), among other sensors. The sensor array 26 is initiated responsive to an operator or machine activation. For instance, the person 16 may activate the real time sensing system 10 (and hence the sensor array 26) upon entering the field 12. As another example, the sprayer machine 18 may activate the real time sensing upon a GPS or other navigational system detecting entry or near entry of a field, signaling to the real time sensing system to cause activation. In some embodiments, the sensor array 26 may always be powered up.

The processor 28 may comprise a computer device, controller, microprocessor, or microcontroller, among other processing devices. In one embodiment, the processor 28 may execute embedded firmware or software stored in a memory, local to or coupled to the processor 28, to process data received by the sensory array 26. For instance, a chemical reaction may occur between the one or more organic chemical compounds received in the air and the sensor materials, resulting in a signal transduction (e.g., change in physical properties, such as a change in the resistance, capacitance, or chemical structure of the chemiresistor or chemicapacitor sensors). The transduction results in a characteristic fingerprint depending on the chemical compound and the makeup of the sensor. In one embodiment, the processor 28 may compare the received fingerprint or fingerprints with a stored fingerprint(s) corresponding to a known emitted organic chemical compound of interest (i.e., of interest in detecting and treating). The processor 28 communicates the detection/identification and other information to the data transfer device 30, which provides feedback of the detection/identification.

The data transfer device 30 may comprise a radio frequency transceiver and antenna for communicating the detection/identification and/or other associated information (e.g., reports, etc.) to another device. For instance, the data transfer device 30 may communicate over a local area network or wide area network, or over other networks (e.g., cell networks, radio frequency channels, etc.). The data transfer device 30 may communicate the detection/identification and/or other information (the detection/identification and other information collectively referred to hereinafter as merely information) to a mobile device (e.g., smartphone, beeper, walkie talkie, cellular phone, etc.), or the real time sensing system 10 may be coupled to a computer located locally (e.g., in the agricultural machine that transports the real time sensing system 10) or transported remotely (e.g., to a computer in a remote office) and the information downloaded/uploaded via a suitable connector to enable the transfer of information. Communications of information may take the form of a text message, among other forms of communications. In some embodiments, the information includes reports or data corresponding to the concentration of the organic compounds, correlated pest infestation, treatments results, etc.

In some embodiments, the data transfer device 30 may provide a control signal that activates an actuator coupled to a sprayer or other device, resulting in an immediate or substantially immediate and selective treatment response from the agricultural machine. In some embodiments, the data communicated by the data transfer device 30 further comprises a time stamp and/or location stamp, such as when the transfer device 30 comprises GPS and/or clock functionality. The data transfer device 30 may also comprise alarm functionality, such as visible, audio, and/or tactile functionality.

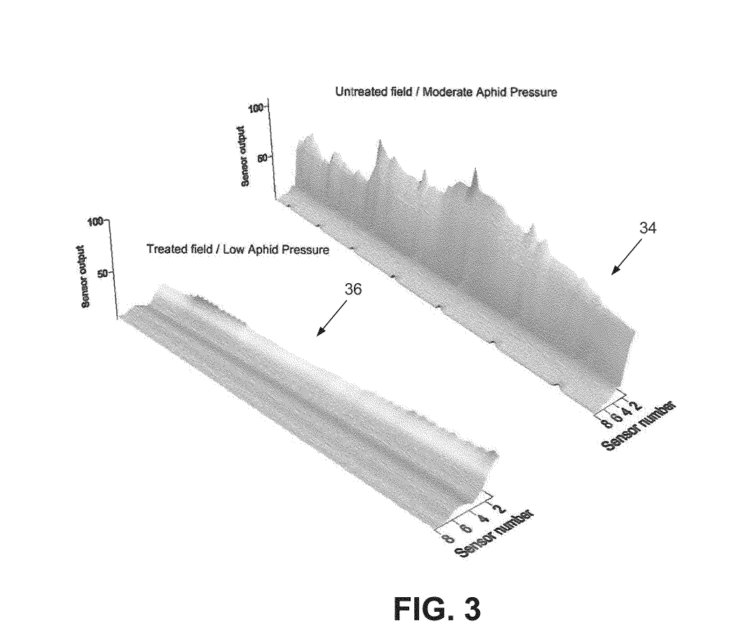

Referring now to FIG. 3, shown are graphics of sensor data provided by an embodiment of the real time sensing system 10. For instance, the data may be communicated to a remote (or local) computer (e.g., laptop, workstation, etc.) or communications device (or displayed using display functionality in the real time sensing system 10 in some embodiments), with a suitable display device for visualization of pre- and post-treatment for an identified area of infestation. The graphic 34 comprises plural axes for data corresponding to a respective sensor output and sensor number for an untreated, moderate aphid pressure field. The graphic 36 comprises data along similar axes for a treated field with low aphid pressure. Such data may be communicated by the data transfer device 30 for an evaluation of how effective was the treatment (e.g., the treatment to remedy the pest infestation). Note that the output of the sensors may be quantified and a threshold value or values set. Further, although aphid-based data is depicted, results for pre- and post-treatment for other pests may be visualized as well.

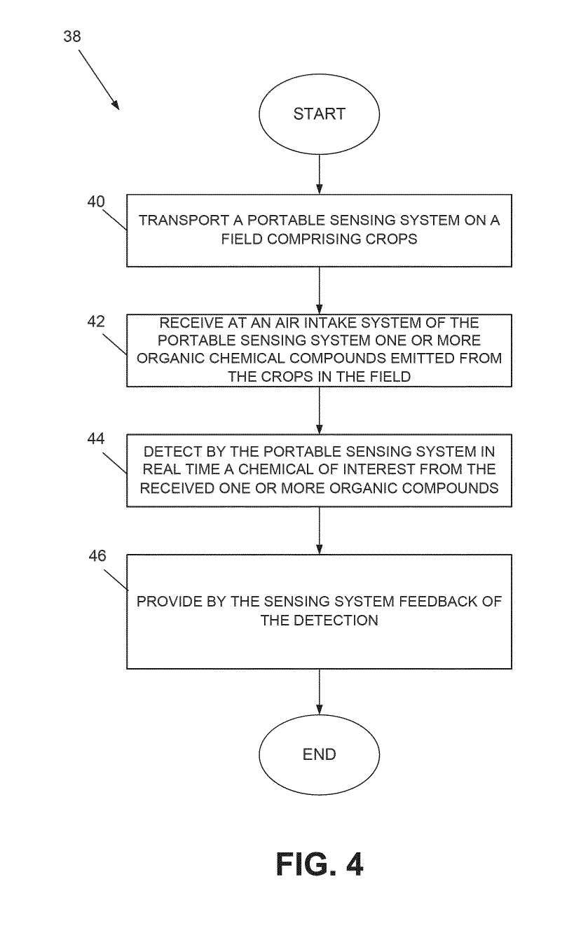

Having described certain embodiments of a real time sensing systems 10, it should be appreciated within the context of the present disclosure that one embodiment of a real time sensing method 38 (e.g., as implemented in one embodiment by the real time sensing system 10, though not limited to the architectures and/or environments depicted in FIGS. 1A-2), illustrated in FIG. 4, comprises transporting a portable sensing system on a field comprising crops (40); receiving at an air intake system of the portable sensing system one or more organic chemical compounds emitted from the crops in the field (42); detecting by the portable sensing system in real time a chemical of interest from the received one or more organic compounds (44); and providing by the sensing system feedback of the detection (46). Such feedback may include the provision of local (and/or remote) alerts, communication to other devices, or activation of certain functionality (e.g., treatment, such as via activation of sprayers).

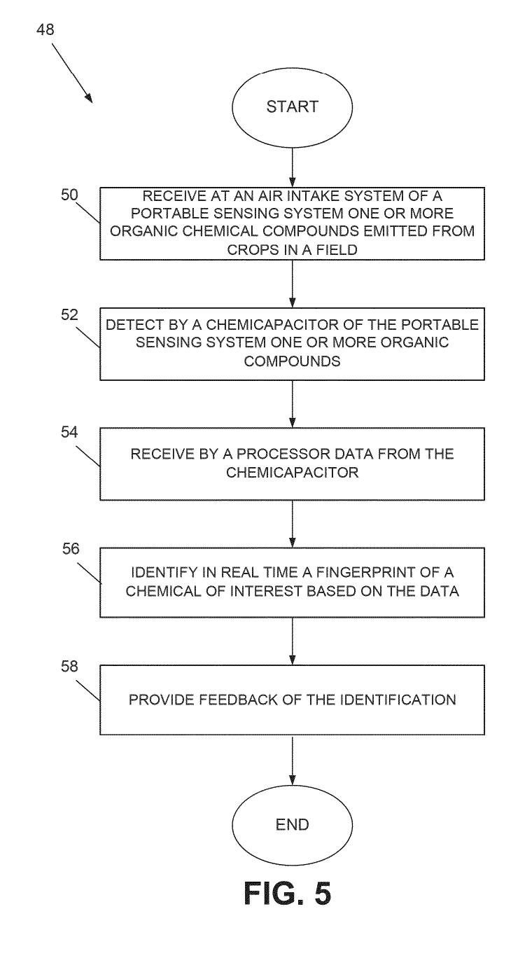

In view of the above description, yet another embodiment of a real time sensing method 48, depicted in FIG. 5, comprises receiving at an air intake system of a portable sensing system one or more organic chemical compounds emitted from crops in a field (50); detecting by a chemicapacitor of the portable sensing system one or more organic compounds (52); receiving by a processor data from the chemicapacitor (54); identifying in real time a fingerprint of a chemical of interest based on the data (56); and providing feedback of the identification (58).

Any process descriptions or blocks in flow diagrams should be understood as merely illustrative of steps performed in a process implemented by a real time sensing system, and alternate implementations are included within the scope of the embodiments in which functions may be executed out of order from that shown or discussed, including substantially concurrently or in reverse order, depending on the functionality involved, as would be understood by those reasonably skilled in the art of the present disclosure.

It should be emphasized that the above-described embodiments of the present disclosure are merely possible examples of implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

At least the following is claimed:

1. A real time sensing method, comprising:
transporting a portable sensing system on a field comprising crops;
receiving at an air intake system of the portable sensing system one or more organic chemical compounds emitted from the crops in the field;
detecting by the portable sensing system in real time a chemical of interest from the received one or more organic compounds; and
providing by the sensing system feedback of the detection.

2. The method of claim 1, wherein transporting comprises securing the portable sensing system on an agricultural machine, the agricultural machine moving on the field.

3. The method of claim 1, wherein transporting comprises securing the portable sensing system on a person, the person walking on the field.

4. The method of claim 1, wherein receiving comprises passively receiving.

5. The method of claim 1, wherein receiving comprises actively receiving.

6. The method of claim 5, wherein actively receiving comprises inducing air carrying the one or more organic compounds using a pump.

7. The method of claim 1, wherein detecting by the portable sensing system comprises detecting by a sensor that is based on chemicapacitor technology.

8. The method of claim 1, wherein detecting comprises determining which of the organic chemical compounds that reacts with sensing materials of the sensing system creates a fingerprint corresponding to a reference fingerprint.

9. The method of claim 1, wherein providing feedback comprises providing an audio alert of the detection.

10. The method of claim 1, wherein providing feedback comprises providing a visible alert of the detection.

11. The method of claim 1, wherein providing feedback comprises providing a tactile alert of the detection.

12. The method of claim 1, wherein providing feedback comprises communicating the detection over a network.

13. The method of claim 1, wherein providing feedback comprises causing selective spraying to automatically be activated on an agricultural machine transporting the portable sensing system.

14. A real time sensing system, comprising:
one or more chemical sensors;
an air induction system configured to cause one or more organic chemical compounds emitted from vegetation in a field to contact the one or more chemical sensors;
a processor configured to receive data from the one or more chemical sensors and identify in real time a fingerprint of a chemical of interest based on the data; and
a data transfer device configured to communicate the identification.

15. The system of claim 14, wherein the air induction system is a passive system.

16. The system of claim 14, wherein the air induction system is an active system.

17. The system of claim 14, wherein the one or more chemical sensors include chemicapacitors.

18. The system of claim 14, wherein the data transfer device is configured to communicate results of treatment of the vegetation relative to the condition of the vegetation prior to the treatment.

19. The system of claim 14, wherein the data transfer device is configured to communicate the identification via a text message.
